(19) United States
(12) Patent Application Publication
(10) Pub. No.: US 2021/0131032 A1
(43) Pub. Date: May 6, 2021

(54) FEEDING SYSTEM AND METHOD FOR FEEDING COMMINUTED CELLULOSIC MATERIAL TO A HIGH-PRESSURE TREATMENT ZONE

(71) Applicant: VALMET AB, Sundsvall (SE)

(72) Inventors: Christer HÄGGLUND, Bergeforsen (SE); Robert WAHLBERG, Sundsvall (SE); Lars FREDRIKSSON, Alnö (SE); Johan CARLSSON, Alnö (SE)

(73) Assignee: VALMET AB, Sundsvall (SE)

(21) Appl. No.: 17/257,078

(22) PCT Filed: Jun. 5, 2019
(86) PCT No.: PCT/SE2019/050524

§ 371 Date: Dec. 30, 2020

(30) Foreign Application Priority Data
Jul. 2, 2018 (SE) 1850833-3

(51) Int. Cl. D21C 7/06; D21C 7/16; B65G 33/22

(52) U.S. Cl. CPC D21C 7/06; C12P 2201/00; B65G 33/22; D21C 7/16

(57) ABSTRACT

The invention relates to a system and a method for feeding comminuted cellulosic material from a low-pressure zone (P_{L}) to a high-pressure zone (P_{E}) with at least 1 bar higher pressure. The invention uses a screw 7 feeder having a feeding screw (1) arranged in a feeding pipe (2). Back blow pulses from the high-pressure zone that may blow 7 backwards from the high-pressure zone (P_{E}) can be ventilated (Vent^{II}) in an intermediate part (P_{c}) of the feeding pipe (2), and thus not reach the low 7-pressure zone.

### BACKGROUND OF THE INVENTION

The invention relates to a feeding system and method for feeding comminuted cellulosic material to a high-pressure treatment zone.

The problem of feeding comminuted cellulosic material to a high-pressure treatment zone lies in the fact that the comminuted cellulosic material is packed randomly and the total void volume in a pile of comminuted cellulosic material approaches well over 50%.

Wood chips stored in an uncompressed state typically show a total void volume exceeding 60%, and the material per se is less suitable for establishment of a pressure plug preventing excess pressure from the high-pressure zone to blow backwards against infeed of chips. The invention is applied in different kind of processes fed with comminuted cellulosic material such as chopped annual plants (bagasse etc.), bamboo, hardwood or softwood.

The high-pressure treatment zone is typically but not limited to a hydrolysis treatment zone where a pressure of about 10 bars is applied at temperatures of about 160-180° C. in weak or strong acidic conditions. A hydrolysis treatment zone is often implemented in new bio-processes where additional products are sought for besides regular pulp for paper production. In hydrolysis carbohydrate is broken into its component sugar molecules by hydrolysis (e.g. sucrose being broken down into glucose and fructose), this is termed saccharification. The sugar molecules extracted may be sold as sweetener or further processed to a variety of products such as ethanol.

The low-pressure treatment zone preceding the high-pressure treatment zone is typically but not limited to a steaming zone for the comminuted cellulosic material where the material is heated from typically ambient temperatures, about 10-30° C., towards higher temperatures established in the high-pressure treatment zones. The heating with steam also serves the purpose to expel both free air between the comminuted material and the air bound in the comminuted material. Often is also the atmospheric steaming followed by pressurized steaming kept at some 1-3 bar higher pressure that elevated the temperature even higher and promotes a more thorough removal of bound air.

### LIST OF DRAWINGS

In the following schematic drawings are details numbered alike in figures, and details identified and numbered in one figure may not be numbered in other figures in order to simplify figures.

FIG. 1a shows a screw feeder in a side view according to prior art;

FIG. 1b shows an alternative screw feeder in a side view according to prior art;

FIG. 1c shows schematically a cross section of the screw feeder in FIG. 1b;

FIG. 2a shows an alternative screw feeder according to prior art as shown in U.S. Pat. No. 3,756,434;

FIG. 2b shows an alternative screw feeder according to prior art as shown in EP 2651794;

FIG. 2c shows an alternative screw feeder according to prior art as shown in U.S. Pat. No. 3,841,465;

FIG. 3a shows a first embodiment of the inventive screw feeder;

FIG. 3b shows a second embodiment of the inventive screw feeder;

FIG. 4a shows a cross-sectional view as seen in X-X in FIG. 3b;

FIG. 4b shows a top view as seen in Y-Y in FIG. 4a;

FIG. 5 shows a control system for the inventive screw feeder;

FIGS. 6a and 6b show schematically proportions between gas and solid matter in comminuted cellulosic material, either in a non-compressed state as shown in FIG. 6a or in a compressed state as shown in FIG. 6b; and

FIG. 7 shows one final layout of an inventive screw feeder, with a partial cross section disclosing the whole feeding screw exposed.

### PRIOR ART

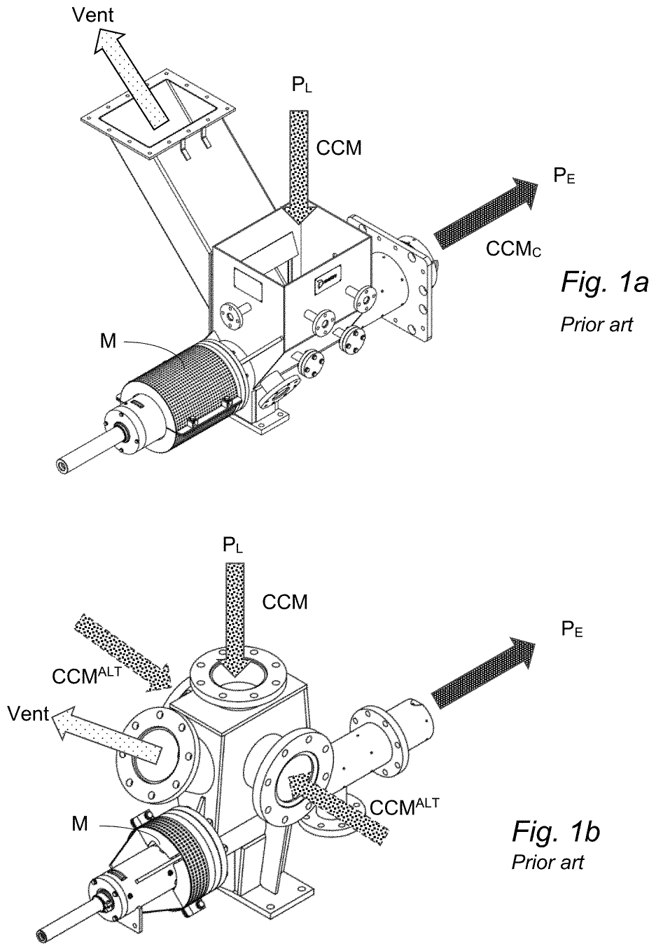

FIG. 1a shows a screw feeder in a side view as sold by Valmet. In this screw feeder is comminuted cellulosic material CCM fed to a feeding chute from a low-pressure zone P_{L}. A feeding screw arranged in the bottom is driven by a motor M and feeds comminuted cellulosic material CCM into the high-pressure treatment zone PE, where the compressed comminuted cellulosic material CCM_{C} leaves the outlet. A venting chute is arranged obliquely upwards from the bottom of the screw housing and is ventilating any excess air that may be blow backwards against the flow of compressed comminuted cellulosic material CCM_{C}.

FIG. 1b shows an alternative screw feeder in a side view as sold by Valmet. In this screw feeder is comminuted cellulosic material CCM fed to a feeding chute from a low-pressure zone P_{L}. A feeding screw arranged in the bottom is driven by a motor M and feeds comminuted cellulosic material CCM into the high-pressure treatment zone PE, where the compressed comminuted cellulosic material CCM_{C} leaves the outlet. A venting chute is arranged obliquely upwards from the bottom of the screw housing and is ventilating any excess air that may be blow backwards against the flow of compressed comminuted cellulosic material CCM_{C}. This embodiment differs from the one shown in FIG. 1a in that alternative inlets, CCM^{ALT}, for the infeed of comminuted cellulosic material CCM, are used which will enable the feeder to be used in different type of process layouts depending on where the flow of comminuted material may come from.

FIG. 1c shows schematically a cross section of the screw feeder in FIG. 1b. As shown here is comminuted cellulosic material CCM fed in from a low-pressure zone P_{L}, typically by gravity but may also use force feed with additional feed screws arranged at an angle to the feeding screw 1. The comminuted cellulosic material CCM piles up at the bottom around the feeding screw 1 that is driven by the motor M. Here is a single flight screw shown in principle, but screws with dual or multiple flights, in parallel or in series, may be used. During transport by the action of the screw 1 is comminuted cellulosic material CCM fed into a feeding pipe 2 at the inlet end 2a thereof and during transport trough the feeding pipe 2 is the comminuted cellulosic material CCM gradually compressed to a compressed state as compressed comminuted cellulosic material CCM_{C} leaves the outlet end 2b. The compression effect, mostly related to the higher pressure in the high-pressure treatment zone P_{E}, is in principle disclosed as successively darker zones in the flow as shown in figure. The comminuted cellulosic material successively compressed in the feeding pipe 2 may establish a pressure plug as the material will create a considerable pressure drop for the gases in the high-pressure zone from blowing backwards and against the flow of cellulosic material. With a sufficient length of the feeding pipe may the screw feeder in established operation create a pressure plug that may withstand a pressure difference of several bars between the low-pressure zone and the high-pressure zone, and thus preventing back blow.

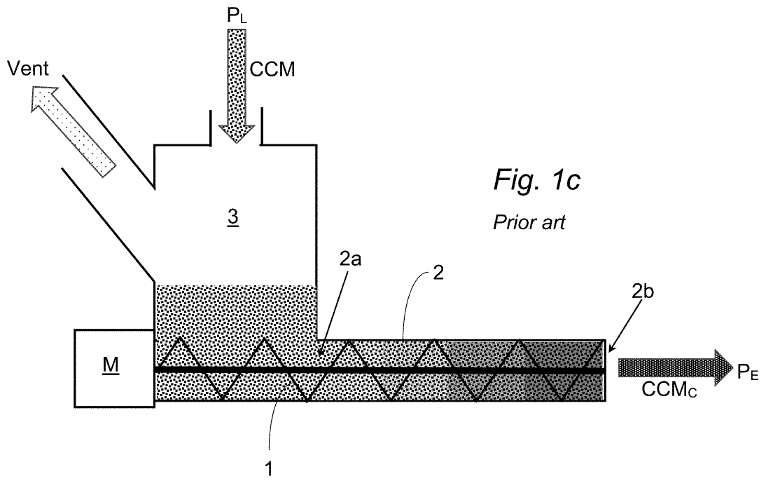

FIG. 2a shows an alternative screw feeder according to prior art as shown in U.S. Pat. No. 3,756,434. In this embodiment is a flow restriction member RM arranged in the outlet end of the feeding pipe 2. The restriction member is biased against the outlet and opens only when the pressure from the compressed plug of material exceeds a certain level. The positioning of the feeding screw in the feeding pipe may be altered as well as the speed depending on the detected pressure in the high-pressure zone.

FIG. 2b shows an alternative screw feeder according to prior art as shown in EP 2651794. However, in this arrangement is the material fed from a high-pressure zone P_{E} to a low-pressure zone P_{L}, and hence the problems are the opposite, preventing high pressure from the preceding high-pressure zone from being wasted.

FIG. 2c shows an alternative screw feeder according to prior art as shown in U.S. Pat. No. 3,841,465. In this implementation are two serially arranged screws 2^{I} and 2^{II} needed, each with its own motor drive M. A pressure plug is established in the first feed screw and the created plug is fed against a restriction member RM assisting in the formation of a compressed plug. The restriction member RM is a cone rotated by a motor M_{RM} with disintegrating members on the conical surface that disintegrate the compacted plug when entering an expansion chamber. The second screw 2^{II} is operated such that no plug is established.

### SUMMARY OF THE INVENTION

The invention is related to an improved system and method for feeding of comminuted cellulosic material where the risk of back blow from a high-pressure zone back to a preceding low-pressure zone is reduced considerably relative know prior art solutions.

The inventive system for feeding comminuted cellulosic material from a low-pressure zone to a high-pressure zone with at least 1 bar higher pressure, comprises:
an inlet chamber connected to the flow of comminuted cellulosic material from a low-pressure zone;
a feeding pipe for feeding the comminuted cellulosic material from the inlet chamber to the high-pressure zone, with an inlet end of said feeding pipe connected to the inlet chamber in the low-pressure zone and an outlet end of said feeding pipe in the high-pressure zone;
a feeding screw arranged in the feeding pipe, driven by a motor such that the comminuted cellulosic material is transported from the inlet end towards the outlet end of the feeding pipe;
a restriction member in the feeding pipe reducing the flow section of the feeding pipe closer to the outlet end of the feeding pipe;
and wherein the wall of the feeding pipe in an intermediate position between the inlet end and the outlet end is equipped with a pressure relief outlet connected to a pressure relief atmosphere with a pressure lower then 0, 5 bar lower than the pressure in the high-pressure zone, this pressure relief atmosphere evacuating any back blow pulses from the high-pressure zone from the flow of comminuted material transported in said feeding pipe before reaching the low-pressure zone and wherein the pressure relief outlet (5) has a regulator (V_{REG}) in the flow section of the pressure relief outlet, regulating the flow of back blow pulses being evacuating from the flow of comminuted material transported in said feeding pipe.

This design of the feeding system enables back blow pulses with gas from the high-pressure zone to be ventilated away before reaching the low-pressure zone. The ventilated gases may be sent to destruction or possibly returned back to the high-pressure zone in order to reduce losses in gas volumes therein. Venting off the back-blow pulses with gas before these gases reach the inlet chamber will also reduce a negative impact on inflow of comminuted cellulosic material into the feeding pipe by the feeding screw, keeping the filling factor of the feeding screw high. Further, emissions of malodourous gases from the high-pressure zone backwards into the low-pressure zone may also be reduced considerably.

According to a preferred embodiment of the invention is the restriction member reducing the flow section of the feeding pipe obtained from a conical form of the feeding pipe having the smallest flow section closer to the outlet end and the largest flow section closer to the inlet end. The conical final part of the feeding pipe will assist in further compression of the comminuted cellulosic material, reducing the overall void volume and create a counter pressure against outflow from the outlet end that creates a denser pressure plug with high pressure loss for gases passing through. In this context is preferably the feeding screw a conical feeding screw with an external diameter corresponding to the conical form of the pipe along its conical extension, thus minimizing leakage flow between the outer edges of the screw flight and the feeding pipe.

According to an alternative preferred embodiment of the invention is the restriction member reducing the flow section of the feeding pipe obtained from a force biased outlet valve arranged in the outlet end of the feeding pipe. The force biased outlet valve of the feeding pipe will assist in further compression of the comminuted cellulosic material, reducing the overall void volume and create a counter pressure against outflow from the outlet end that creates a denser pressure plug with high pressure loss for gases passing through. The force biased outlet valve may also physically close the outlet end of the feeding pipe if a shortage in feeding of comminuted cellulosic material to the inlet chamber is experienced.

According to a further preferred embodiment of the invention is the intermediate position of the pressure relief outlet located at a distance from the outlet end exceeding at least one full turn of a flight on the feeding screw. This prevents a straight axial back blow of gases through the plug, as gases must follow the screw flight surface. Preferably is the intermediate position of the pressure relief outlet located at a distance from the outlet end exceeding at least 50 centimeters. In principle is a more effective pressure plug created with longer distance, in the range 50-100 centimeters, but costs for the feeding screw increases in proportion to length, so the distance chosen is a tradeoff between pressure plug requirements and costs for the feeding system.

According to yet a further preferred embodiment of the invention is preferably the intermediate position of the pressure relief outlet located at a distance from the inlet end exceeding at least one half turn of a flight on the feeding screw. The screw flights will thus assist in preventing back blow of gas from the intermediate position and backwards towards the inlet chamber in the low-pressure zone. The numbers of turns of the flight may be greater, i.e. between 1-3 turns. In aspects of distance may the intermediate position of the pressure relief outlet be located at a distance from the inlet end exceeding at least 20 centimeters, and preferably in the range 50-100 centimeters,

According to a further preferred embodiment of the invention is the pressure relief outlet equipped with a screen member at the entry of the pressure relief outlet, i.e. in level with the wall of the feeding pipe, preventing expansion of the plug of comminuted material into the pressure relief outlet. The early formation of first phases of the pressure plug ahead of the intermediate position, will thus not be wasted as the first formation of the pressure plug will stay intact during passage of the intermediate position where ventilation occurs.

According to a further preferred embodiment of the invention is the pressure relief outlet equipped with a regulator in the flow section of the pressure relief outlet, regulating the flow of excess air being evacuating from the flow of comminuted material transported in said feeding pipe. The regulator may preferably be connected to a control unit adjusting the conditions in the pressure relief outlet, using at least one pressure sensor connected to the control unit and with at least one pressure sensor located in the pressure relief outlet and optionally at least one more sensor in low-pressure zone or one more sensor in the high-pressure zone. The order of evacuation may thus be altered automatically in a teed-back manner depending on operational conditions of the feeding system.

The regulator may be an adjustable restriction valve connected to atmosphere in the simplest embodiment, if for example the pressure in the low-pressure zone is 2-3 bars, and the intermediate pressure somewhat higher. The restriction valve may then be connected to atmosphere and the flow rate in the pressure relief outlet increased by opening of the restriction, and when decreasing the flow rate in the pressure relief outlet reduced by closing the restriction gradually up until the point where the restriction is totally closed, and no flow is developed in the pressure relief outlet. Alternatively, the regulator may be an adjustable blower with variable evacuation capacity, either a rpm-controlled pump with evacuation flow increasing with rpm, or a pump with variable geometry.

The method for feeding comminuted cellulosic material from a low-pressure zone to a high-pressure zone with at least 1 bar higher pressure, comprising following steps:
filling an inlet chamber with a flow of comminuted cellulosic material from a low-pressure zone:
feeding the comminuted cellulosic material from the inlet chamber to the high-pressure zone with a motor driven feeding screw located in a feeding pipe, with an inlet end of said feeding pipe connected to the inlet chamber in the low-pressure zone and an outlet end of said feeding pipe in the high-pressure zone; such that the comminuted cellulosic material is transported from the inlet end towards the outlet end of the feeding pipe;
arranging a restriction member in the feeding pipe reducing the flow section of the feeding pipe closer to the outlet end of the feeding pipe;
   wherein back blow pulses from the high-pressure zone are evacuated in an intermediate position from said feeding pipe between the inlet end and the outlet end of said feeding pipe using a pressure relief outlet located in the wall of the feed pipe wherein the flow in the pressure relief outlet is regulated and thus regulating the flow of back blow pulses from the high-pressure zone being evacuated from the flow of comminuted material transported in said feeding pipe.

If these method steps are implemented will an advantageous prevention of back blow from the high-pressure zone be obtained, said back blow reaching the low-pressure zone and disturbing the filling of the feeding screw.

The inventive method may preferably include forming a compressed plug flow in the pipe after the intermediate position of the pressure relief outlet, said plug flow having a length preventing axial back blow of gases from the high-pressure zone through the compressed plug flow. The inventive method may preferably include establishing an increased pressure drop for back blow of gases from the high-pressure zone through the compressed plug flow.

The inventive method may also include forming a compressed plug flow in the pipe before the intermediate position of the pressure relief outlet, said plug flow having a length preventing axial back blow of gases from the intermediate position through the compressed plug flow. The inventive method may preferably include establishing an increased pressure drop for back blow of gases from the intermediate position of the pressure relief outlet towards the inlet chamber through the compressed plug flow.

The inventive method may also include preventing the compressed plug flow from expanding when passing the pressure relief outlet using a screen member at the entry of the pressure relief outlet 5, i.e. in level with the wall of the feeding pipe.

The inventive method may also include regulating the flow in the pressure relief outlet and thus regulating the flow of excess air being evacuating from the flow of comminuted material transported in said feeding pipe.

The inventive method may also include that the regulation is made dependent on the pressure conditions in the pressure relief outlet, with at least one pressure detection in the pressure relief outlet and optionally at least one more pressure detection in the low-pressure zone or one more pressure detection in the high-pressure zone.

The inventive method may also include that the regulation is made using an adjustable restriction.

The inventive method may also include that the regulation is made using an adjustable evacuator with variable evacuation capacity.

### DETAILED DESCRIPTION OF THE INVENTION

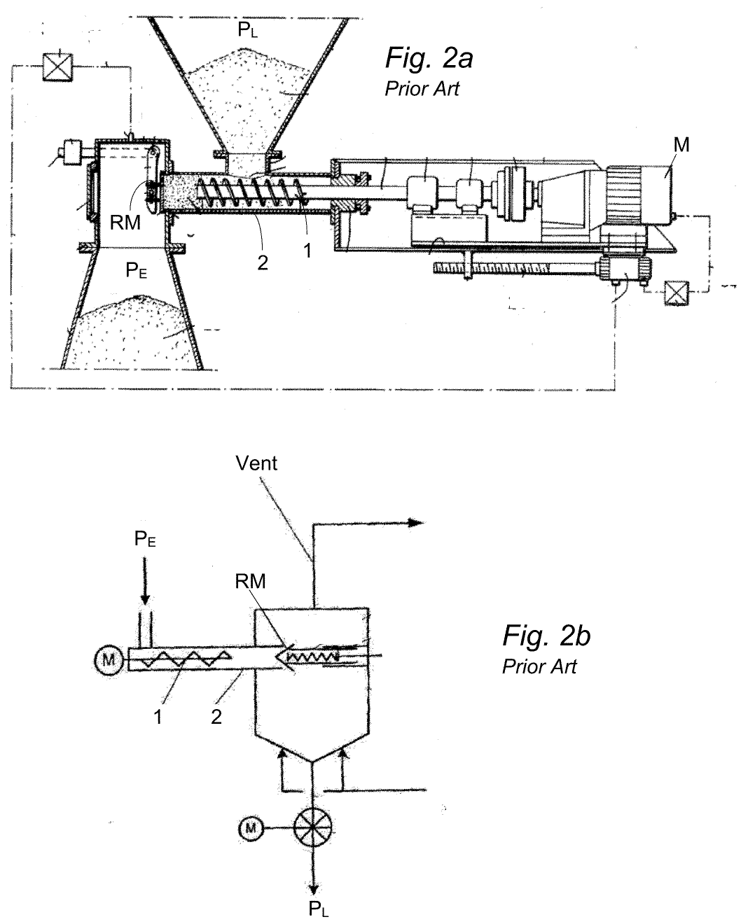

In FIG. 3a is shown a first embodiment of the inventive screw feeder. Most features are already shown and described in relation to FIG. 2, and hence are only the modifications made described. In this embodiment in a pressure relief outlet 5 arranged in the feeding pipe in a position between the inlet end 2a and the outlet end. In this embodiment is also a ventilation duct arranged in the inlet chamber, but it should be clear that in some applications may this ventilation duct arranged in the inlet chamber be omitted. A restriction member RM is arranged in the outlet end reducing the flow section of the feeding pipe. Here a spring biased conical plug that pushes the closing cone towards a closing position. This closing cone may be motor driven in the same way as shown in FIG. 3c, but in the simplest form as shown here it may be non-revolving. The intermediate position PC of the pressure relief outlet 5 is located at a distance B from the outlet end 2b exceeding at least one full turn of a flight on the feeding screw 1. In this example dose to 1,5 turns. The intermediate position of the pressure relief outlet 5 is preferably located at a distance B from the outlet end 2b exceeding at least 50 centimeters. The exact distance needed depends on size and form and compressibility of the comminuted cellulosic material, which necessary distance may differ significantly between hardwood and softwood chips, as well as to form if chopped annual plants are fed. Irrespective of type of material is a pressure profile developed in the feeding pipe where the pressure gradually is reduced in the feeding pipe 2 towards the inlet end 2a.

Further, wherein the intermediate position PC of the pressure relief outlet 5 is located at a distance A from the inlet end 2a exceeding at least one half turn of a flight on the feeding screw, and in this figure in excess of 1 turn. The intermediate position of the pressure relief outlet 5 is preferably located at a distance A from the inlet end 2a exceeding at least 20 centimeters.

In FIG. 3b is shown a second embodiment of the inventive screw feeder. In comparison to FIG. 3a is another type of restriction member RM used. The restriction member RM reducing the flow section of the feeding pipe is obtained from a conical form of the feeding pipe 2 having the smallest flow section closer to the outlet end and the largest flow section closer to the inlet end. The complementing feeding screw 1 is preferably a conical feeding screw with an external diameter corresponding to the conical form of the feeding pipe 2 along its conical extension. Even tough FIG. 3a shows one principle type of restriction member, and FIG. 3b shows another principle type of restriction member, it should be clear that both of these restriction members may be used in a feeding system, both types of restriction members contributing in forming a denser pressure plug.

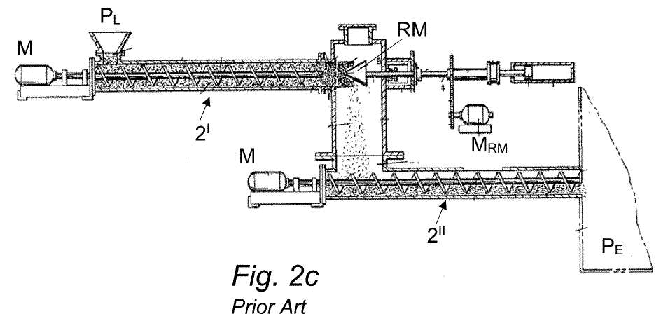

FIG. 4a shows a cross-sectional view through the feeding pipe 2 as seen in the view X-X in FIG. 3b.

FIG. 4b shows a top view Y-Y in FIG. 4a. The pressure relief outlet 5 has a screen member 4 at the entry of the pressure relief outlet 5, i.e. in level with the wall of the feeding pipe 2, preventing expansion of the plug of comminuted material into the pressure relief outlet. In this example is the screen configured with continuous slots running in in the axial direction of the feeding pipe as seen in FIG. 4b. Straight slots may be preferred as these slots may be exposed to a rubbing action from the passing plug of comminuted material as well as the passing flights of the feeding screw, keeping the slots open. As is well known from digester screens may the slots have an open downstream end that allows comminuted cellulosic material to leave the slot even if it is partially pushed into the slot. Such an open end of the slot may be obtained by a small step-out (not shown) in the feeding pipe having a step-out size of 5-15 millimeter at the very downstream end of the slots. However, other type of ventilation holes may be used, for example grating dimples or slanted round holes with drilled holes at a sharp angel versus the flow of the plug.

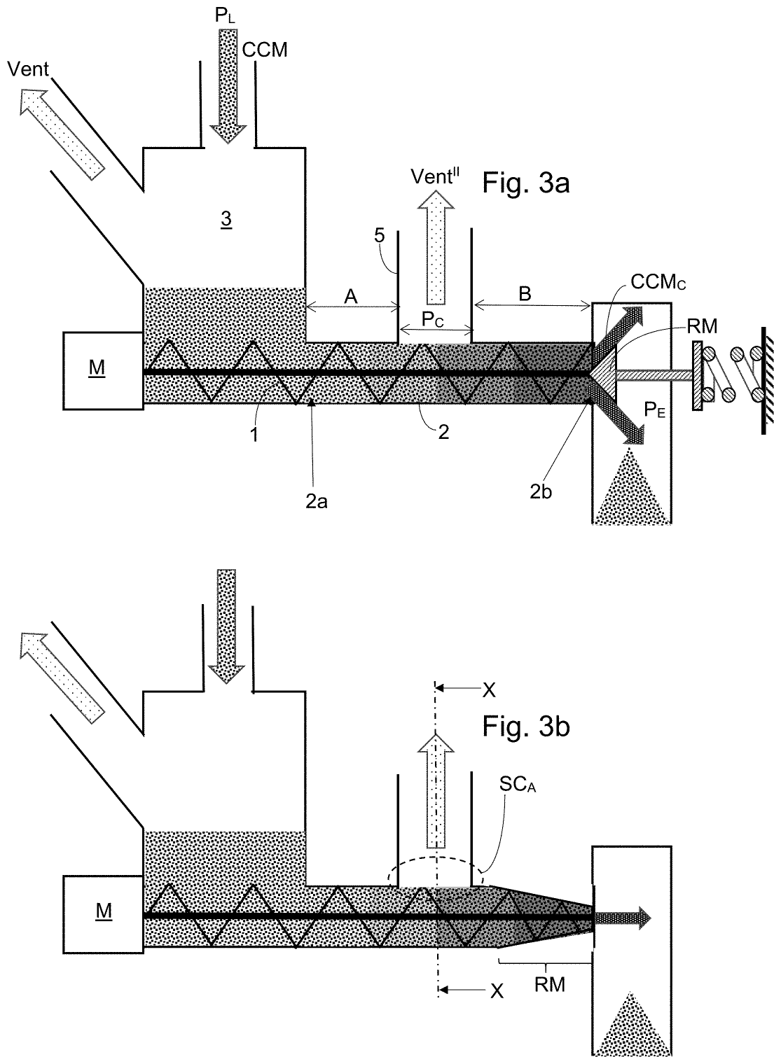

FIG. 5 shows schematically a control system for the inventive screw feeder. The pressure relief outlet 5 may have a regulator V_{REG} in the flow section of the pressure relief outlet, regulating the flow of excess air being evacuated from the flow of comminuted material transported in said feeding pipe.

The regulator V_{REG} is connected to a control unit CPU adjusting the conditions in the pressure relief outlet, using at least one pressure sensor connected to the control unit and with at least one pressure sensor P₂ located in the pressure relief outlet 5 and optionally at least one more sensor P₁ in the low-pressure zone P_{L} or one more sensor P₃ in the high-pressure zone P_{L}. In the simplest closed-loop control may only the pressure in the pressure relief outlet be used to control the regulator, maintaining the pressure at any selected predetermined level. Additional sensors in the low- and high-pressure zone may be used to adjust the regulator if sudden changes in the low- and/or high-pressure zone may call for changes in the regulator ahead of detected changes in the pressure relief outlet which typically occurs at some time delay. Different kinds of regulators V_{REG} may be used and in the simplest embodiment could an adjustable restriction valve connected to atmosphere be used as the regulator.

As the pressure pulse that may penetrate the plug comes from the high-pressure zone, could this pressure pulse at higher pressure simply be vented to atmosphere. Alternatively, the regulator V_{REG} could be an adjustable blower with variable evacuation capacity. A blower or pump may even establish a lower pressure than ambient pressure in the pressure relief outlet.

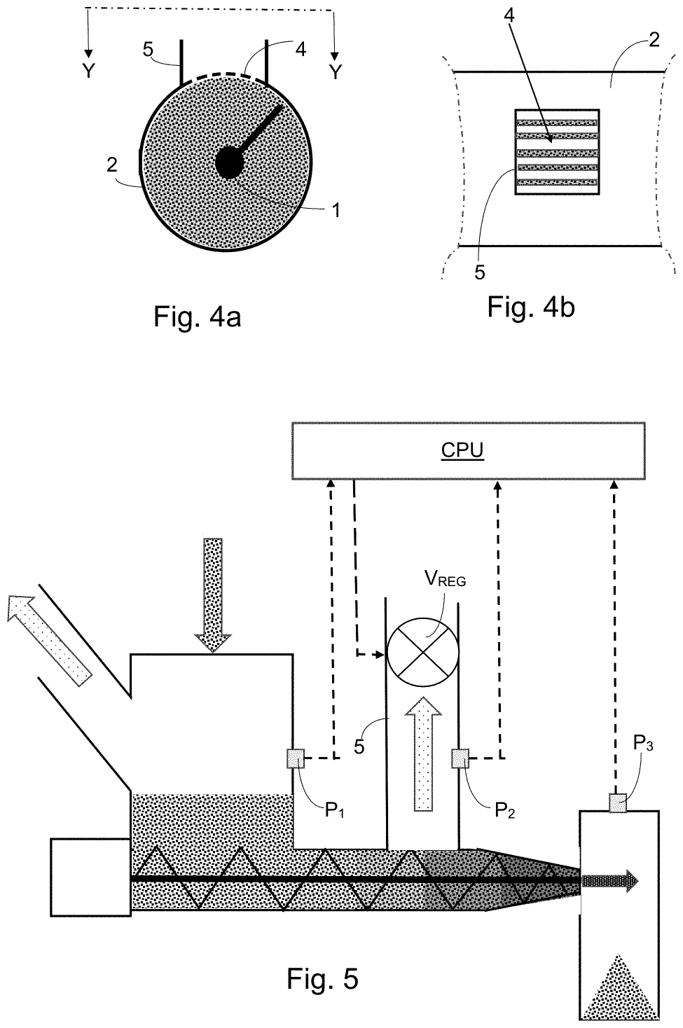

In order to visualize the working conditions for the feeding system, more or less the root cause for the problem of back blow when feeding comminuted cellulosic material to a high-pressure zone, are the volumetric proportions between gas and solid matter in comminuted cellulosic material schematically shown in FIGS. 6a and 6b. FIG. 6a show the volumetric proportions in a non-compressed state, i.e. wood chips stored in I pile, and in FIG. 6b are the volumetric proportions in a compressed state shown.

FIG. 6b roughly indicating the practical limit for compressing comminuted cellulosic material in a plug screw feeder. In some extreme plug screw feeders may the volumetric proportion of solids exceed ⅔ of the total volume (about 66%) but then at expense of high operating costs and increased wear in the plug screw. Additional compression may also be obtained with large press rams or press rolls, but then at expense of dramatic increase of investment costs. As indicated here may still a total void volume between the comminuted material amount to ⅓ (about 33%) in a compressed state, and this could not establish a perfect pressure plug as the pressurized gases will leak trough the plug, but then at expense of pressure drop when passing the material. This creates a pressure profile that drops as seen from the outlet end of the feeding pipe. The actual pressure profile differs for differing cellulosic material being transported, but the skilled person could in a laboratory find the boundaries for the distances necessary for implementing the invention (see the distances A and B in FIG. 3a). But as a rule, the necessary distances A and B will decrease in proportion to:
reduced size in the comminuted cellulosic material (dust vs regular chips)
reduced stiffness of the comminuted cellulosic material (softwood vs hardwood, or effects from preceding treatment in low pressure zone, i.e. steaming or soaking processes).

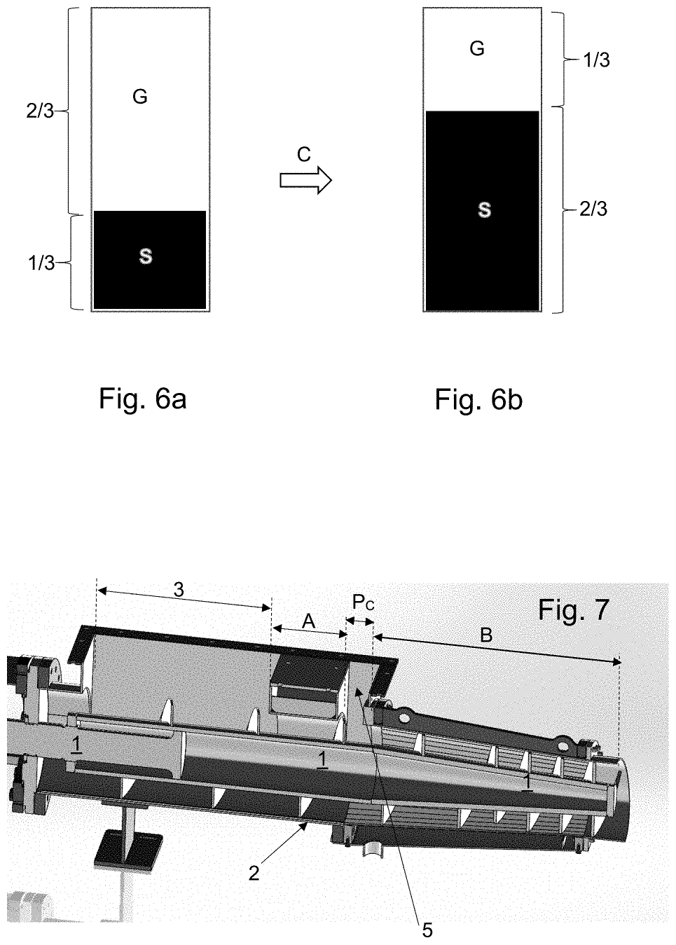

In FIG. 7 is a prototype of the invention shown. In this final layout of an inventive screw feeder, is a conical plug screw feeder used with one single flight with variable pitch along the axial feeding direction. The flight turns are closer at the outlet where the compression effect is needed the most, and the flight turns in the inlet chamber are located at a longer distance apart. The flight exposed in the inlet chamber 3 should only feed the material to the inlet end of the feeding pipe 2, at low pressure conditions, and once the material enters the feeding pipe starts compression. The pressure relief outlet 5 is located at the distance A after the inlet end of the feeding pipe and establish the pressure control zone P_{C}. After the pressure control zone starts the final compression of a pressure plug over the distance B in the conical part of the feeding screw.

1.-21. (canceled)

22. A system for feeding comminuted cellulosic material from a low-pressure zone to a high-pressure zone with at least 1 bar higher pressure, comprising:
An inlet chamber connected to the flow of comminuted cellulosic material from a low-pressure zone;
a feeding pipe for feeding the comminuted cellulosic material from the inlet chamber to the high-pressure zone, with an inlet end of said feeding pipe connected to the inlet chamber in the low-pressure zone and an outlet end of said feeding pipe in the high-pressure zone;
a feeding screw arranged in the feeding pipe, driven by a motor such that the comminuted cellulosic material is transported from the inlet end towards the outlet end of the feeding pipe;
a restriction member in the feeding pipe reducing the flow section of the feeding pipe closer to the outlet end of the feeding pipe; characterized in that the wall of the feeding pipe in an intermediate position between the inlet end and the outlet end is equipped with a pressure relief outlet connected to a pressure relief atmosphere with a pressure lower then 0.5 bars lower than the pressure in the high-pressure zone, this pressure relief atmosphere evacuating any back blow pulses from the high-pressure zone from the flow of comminuted material transported in said feeding pipe before reaching the low-pressure zone and, wherein the pressure relief outlet has a screen member at the entry of the pressure relief outlet, i.e. in level with the wall of the feeding pipe, preventing expansion of the plug of comminuted material into the pressure relief outlet.

23. A system according to claim 22, wherein the restriction member reducing the flow section of the feeding pipe is obtained from a conical form of the feeding pipe having the smallest flow section closer to the outlet end and the largest flow section closer to the inlet end.

24. A system according to claim 23, wherein the feeding screw is a conical feeding screw with an external diameter corresponding to the conical form of the pipe along its conical extension.

25. A system according to claim 22, wherein the restriction member reducing the flow section of the feeding pipe is obtained from force biased outlet valve arranged in the outlet end of the feeding pipe.

26. A system according to claim 22, wherein the intermediate position of the pressure relief outlet is located at a distance from the outlet end exceeding at least one full turn of a flight on the feeding screw.

27. A system according to claim 26, wherein the intermediate position of the pressure relief outlet is located at a distance from the outlet end exceeding at least 50 centimeters.

28. A system according to claim 22, wherein the intermediate position of the pressure relief outlet is located at a distance from the inlet end exceeding at least one half turn of a flight on the feeding screw.

29. A system according to claim 28, wherein the intermediate position of the pressure relief outlet is located at a distance from the inlet end exceeding at least 20 centimeters.

30. A system according to claim 22, wherein the pressure relief outlet has a regulator in the flow section of the pressure relief outlet, regulating the flow of back blow pulses being evacuating from the flow of comminuted material transported in said feeding pipe.

31. A system according to claim 30, wherein the regulator is connected to a control unit adjusting the conditions in the pressure relief outlet, using at least one pressure sensor connected to the control unit and with at least one pressure sensor located in the pressure relief outlet and optionally at least one more sensor in low-pressure zone or one more sensor in the high-pressure zone.

32. A system according to claim 30, wherein the regulator is an adjustable restriction valve 20 connected to atmosphere.

33. A system according to claim 30, wherein the regulator is an adjustable blower with variable evacuation capacity.

34. A method for feeding comminuted cellulosic material from a low-pressure zone to a high-pressure zone with at least 1 bar higher pressure, comprising following steps:
filling an inlet chamber with a flow of comminuted cellulosic material from a low-pressure zone;
feeding the comminuted cellulosic material from the inlet chamber to the high-pressure zone with a motor driven feeding screw located in a feeding pipe, with an inlet end of said feeding pipe connected to the inlet chamber in the low-pressure zone and an outlet end of said feeding pipe in the high-pressure zone; such that the comminuted cellulosic material is transported from the inlet end towards the outlet end of the feeding pipe;
arranging a restriction member in the feeding pipe reducing the flow section of the feeding pipe closer to the outlet end of the feeding pipe; characterized in that evacuating back blow pulses from the high-pressure zone from said feeding pipe in an intermediate position between the inlet end and the outlet end of said feeding pipe using a pressure relief outlet located in the wall of the feed pipe, further comprising preventing expansion of the plug of comminuted material into the pressure relief outlet by means of a screen member at the entry of the pressure relief outlet, i.e. in level with the wall of the feeding pipe.

35. A method according to claim 34, wherein forming a compressed plug flow in the pipe after the intermediate position of the pressure relief outlet, said plug flow having a length preventing axial back blow of gases from the high-pressure zone through the compressed plug flow.

36. A method according to claim 34, wherein forming a compressed plug flow in the pipe after the intermediate position of the pressure relief outlet, said plug flow establishing an increased pressure drop for back blow of gases from the high-pressure zone through the compressed plug flow.

37. A method according to claim 34, wherein forming a compressed plug flow in the pipe before the intermediate position of the pressure relief outlet, said plug flow having a length preventing axial back blow of gases from the intermediate position through the compressed plug flow.

38. A method according to claim 34, wherein forming a compressed plug flow in the pipe before the intermediate position of the pressure relief outlet, said plug flow having a length establishing an increased pressure drop for back blow of gases from the intermediate position of the pressure relief outlet through the compressed plug flow.

39. A method according to claim 34, wherein the flow in the pressure relief outlet is regulated and thus regulating the flow of back blow pulses from the high-pressure zone being evacuated from the flow of comminuted material transported in said feeding pipe.

40. A method according to claim 39, wherein the regulation is made dependent on the pressure conditions in the pressure relief outlet, with at least one pressure detection in the pressure relief outlet and optionally at least one more pressure detection in the low-pressure zone or one more pressure detection in the high-pressure zone.

41. A method according to claim 39, wherein the regulation is made using an adjustable restriction.

42. A method according to claim 39, wherein the regulation is made using an adjustable evacuator with variable evacuation capacity.
